(19) United States
(12) Patent Application Publication
(10) Pub. No.: US 2023/0248627 A1
(43) Pub. Date: Aug. 10, 2023

(54) COMPOSITION FOR HAIR FOLLICLE MODULATION, METHODS AND USES THEREOF

(71) Applicant: Universidade do Minho, Braga (PT)

(72) Inventors: Artur Manuel CAVACO PAULO, Braga (PT); Maria Teresa GONÇALVES DE MACEDO MATAMÁ, Paços de Ferreira (PT); Cristiana Filipa CHAVES COSTA, Gondifelos (PT); Bruno PACHECO FERNANDES, Moreira de Cónegos (PT)

(21) Appl. No.: 18/003,129

(22) PCT Filed: Jun. 28, 2021
(86) PCT No.: PCT/IB2021/055750

§ 371 Date: Dec. 22, 2022

(30) Foreign Application Priority Data
Jun. 26, 2020 (PT) 116531

(51) Int. Cl. A61K 8/49; A61K 8/44; A61Q 5/10; A61Q 5/04; A61K 8/42

(52) U.S. Cl. CPC A61K 8/4953; A61K 8/42; A61K 8/445; A61K 8/498; A61Q 5/04; A61Q 5/10; A61K 2800/48; A61K 2800/56; A61K 2800/524

(57) ABSTRACT

Abstract: The present disclosure relates to a composition for use in the hair follicle modulation comprising a bioactive agent a thickener agent; a preservative; and a denaturing alcohol. The present disclosure also comprises a pharmaceutical composition for use in the prevention, therapy or treatment of abnormal defective production of melanin, comprising a bioactive agent selected from dipyridamole, dipyridamole salt, rivastigmine, rivastigmine salt, paroxetine, paroxetine salt, or mixtures thereof. The use of said bioactive agent as a hair modulator is also encompassed.

### TECHNICAL FIELD

The present disclosure relates to the topical use of a group of modulation bioactive agents with known toxicological profile, in order to safely alter the hair’s properties, namely the colour and shape without compromising the physical strength, of the new hair as it is produced/synthesized in the hair bulb cells.

### BACKGROUND

The scalp hair of each individual is distinctive, both in terms of growth rate and natural physical properties as colour and shape. Scalp hair is a highly perceptible feature with great individual and social impacts, frequently considered an indicative of cultural identity, personal style or health, and a source of information for biological and forensic analyses [1].

Many individuals are willing to achieve their own idea of hair beauty through the desired and idealized hairstyling, which changes hair fibre physical features: the length, colour or shape [2]. Consequently, there is a vast global hair care cosmetic industry feeding this increasing market. Regarding the year of 2016 and the forecast for 2017-2024 posted by Inkwood Research (Boston, MA, USA), the market for cosmetic hair products constitutes 15% of the global market for personal care and beauty products. In terms of type of hair product, approximately 6% of the market is curling/relaxation products and 16% is colouring products.

All human hair fibres typically have the same basic structure. Their structure is built and shaped by the hair follicle, a self-sufficient and highly organized mini-organ with both proliferating (dividing) and differentiating (functional/specialized) cell compartments. Hair colour and shape are determined in the hair follicle, specifically in the bulb, as a direct result of the organization of its various structural elements, being the proteins the most significant element [2,3].

Hair colour is determined by the amount, type and distribution of melanin. Melanin is a complex mixture of biopolymers that are synthesized by specialized cells called melanocytes.

Currently, the change of natural colour is carried out through chemical staining procedures. Over 70% of hair dye products in Europe are based on permanent hair dyes, which include oxidation-reduction reactions [4]. Permanent dyes are produced directly in the hair fibre from colourless precursors (developer and coupler) through a chemical reaction in the presence of hydrogen peroxide (H₂O₂) as an oxidizing agent. Different concentrations of hydrogen peroxide and precursor agents produce a variety of colours. Higher concentrations of hydrogen peroxide can bleach the hair’s natural pigment, so the oxidizing step works both in lightening and in colour production.

The production of the highly complex biomaterial (the hair fibre) in the hair follicle, and how shape is established is a topic that remains relatively unexplained. Five possible curl mechanisms have been proposed that can coexist: asymmetric expression of structural keratins in the pre-cortex, variable cortical cell shape and keratin filament orientation in relation to the axis of hair growth, asymmetric proliferation in cells forming the inner and outer root sheaths, polymorphisms in inner root sheath proteins link to variation in fibre shape and dermal papilla asymmetry [5].

Permanent treatments for straightening or waving hair depend on the application of a reducing agent (thioglycolic or thiolatic acid, for example) and an alkaline agent (ammonia, monoethanolamine, ammonium hydrogen carbonate, for example), which promotes cleavage (reduction) of disulfide bonds which are naturally present in the proteins that make up the hair [6-8]. The most aggressive straightening treatments are used in African hair. Relaxing this type of hair is done with a pH higher than 12, using caustic soda. In addition, it is necessary to apply an external physical force to achieve the desired shape of the hair, followed by exposure to an oxidizing agent (sodium bromate or hydrogen peroxide) or, in more recent formulations, only exposure to air, so that disulfide bonds can re-establish (oxidation) in the new positions. These aggressive oxidative treatments that occur at alkaline pH lead to damage on the surface and cortex of hair fibres affecting porosity, smoothness and shine, as well as the mechanical properties [9]. In African hair straightening treatments, up to 60% loss of fibre strength/resistance frequently occurs as well as scalp irritation (burns and contact dermatitis) [10].

Summing up, the current cosmetic procedures for obtaining different hair colours and shapes rely on alkaline emulsions and/or strong oxidative-reducing power, that are applied directly on hair fibres. The chemical processes in the basis of the current hair treatments to modify the colour and shape aim to permanently alter the cortex of the hair fibre [4,6-7]. However, the frequent use of these cosmetic procedures has adverse side effects for consumers, more specifically, it induces hair fibre and scalp damage. Despite that, over the years, there has been little evolution in these traditional cosmetic technologies.

Changes in colour and shape of the hair have been reported as a side effect of pharmacological drugs used in the treatment of several diseases. Drug-induced changes in hair colour are the most common and can be either as lightening or darkening. Although scarcer, drug-induced changes in hair shape as straightening and curling or kinking have also been reported (Table 1). These side effects seem to be related to the modulation of molecular determinants of hair colour (namely, melanin) or shape in the hair follicle.

The modulation of melanogenesis in hair follicles has become an interesting alternative to the conventional cosmetic methods of hair coloration. Some patents have already claimed the topical use of melanogenesis modulators in cosmetic or pharmaceutical compositions to promote a change in the natural colour of hair fibres.

Document US 5273739 (Baral, 1993, Composition and treatment for darkening hair colour) describes a method of darkening the hair colour comprising tretinoin application to the scalp.

Document WO 98/24407 (Duranton et al., 1998, Use of paracetamol as depigmenting agent) describes the use of paracetamol in a composition as depigmenting and/or bleaching agent for human hair.

TABLE 1

List of pharmacological drugs reported to change natural hair colour and/or shape as a side effect of medical treatments

| Drug | Reason for use | Hair change | Reference |
|---|---|---|---|
| Acitretin | Psoriasis | Darkening | [11,12] |
| | | Curling | |
| | | Kinking | [13] |
| Alitretinoin | Severe chronic hand eczema | Curling | [14] |
| Atezolizumab | Lung Cancer | Darkening | [15] |
| Chloroquine | Malaria prophylaxis | Lightening | [16] |
| | Dermatomyositis | | [17] |
| Cisplatin | Metastatic germ cell neoplasm of the testis | Darkening | [18] |
| | | Lightening | |
| | | Straightening | |
| | | Curling | |
| Cyanocobalamine | Pernicious anaemia | Darkening | [19] |
| Cyclosporin | Psoriasis | Darkening | [20] |
| Dabrafenib | Metastatic melanoma | Lightening | [21] |
| | | Kinking | |
| Dasatanib | Chronic myeloid leukemia | Lightening | [22] |
| Defibrotide | Deep venous thrombosis | Darkening | [23] |
| Erlotinib | Adenocarcinoma of the lung | Darkening | [24] |
| Etretinate | Palmo plantar keratoderma | Kinking | [25] |
| | Psoriasis | | |
| | | Lightening | [26] |
| | | Darkening | [27] |
| | Pityriasis rubra pilaris | | [28] |
| Gefitinib | Non-small-cell lung cancer | Curling | [29] |
| Hydroxychloroquine | Discoid lupus erythematosus | Lightening | [30] |
| Imatinib mesylate | Gastrointestinal stromal tumor | Lightening | [31] |
| | Chronic myeloid leukemia | | [32] |
| L-Thyroxine | Hypothyroidism | Darkening | [33] |
| Latanoprost | Open-angle glaucoma | Darkening | [34] |
| Lenalidomide | Multiple myeloma | Darkening | [35] |
| Levodopa | Parkinson’s disease | Darkening | [36] |
| Nivolumab | Lung Cancer | Darkening | [15] |
| para-aminobenzoic acid | Lymphoblastoma cutis | Darkening | [37] |
| | Dermatomyositis | | |
| | Dermatitis herpetiformis | | |
| | Scaly erythroderma | | |
| | Scleroderma | | |
| Pazopanib | Hurthle cell carcinoma | Lightening | [38] |
| | Myofibroblastic sarcoma | | [39] |
| Pembrolizumab | Lung Cancer | Darkening | [15] |
| Perampanel | Epilepsy (Pitt Hopkins syndrome) | Curling | [40] |
| Prednisone | Bullous pemphigoid | Darkening | [41] |
| Sunitinib malate | Gastrointestinal stromal tumor | Lightening | [42] |
| Tamoxifen | Breast cancer | Darkening | [43] |
| Thalidomide | Multiple myeloma | Darkening | [44] |
| Triptorelin | Precocious puberty | Lightening | [45] |
| Valproic acid | Seizure | Lightening | [46] |
| | Bipolar disorder | Curling | [47] |
| Vemurafenib | Metastatic melanoma | Darkening | [21] |
| | | Kinking | |
| Verapamil | Hypertension | Darkening | [48] |
| α-Interferon | Melanoma | Lightening | [49] |
| α-Interferon+ Ribavirin | Chronic Hepatitis C | Straightening | [50] |

Document EP 0655907 B1 (Gilchrest et al., 1999, Use of diacylglycerols for increasing the melanin content in melanocytes) describes a cosmetic method to darkening human hair, using a topical formulation comprising diacylglycerols as inducers of melanin synthesis in melanocytes present in hair follicles.

Document US 6365135 B1 (Philippe et al., 2002, Use of amino phenolamide derivatives as depigmentation agents) describes the use of aminophenol amide derivatives in compositions as an agent for depigmenting and/or bleaching of human body hairs and/or head hair.

Document US 6551581 B1 (Mahalingam et al., 2003, Methods for improving the aesthetic appearance of skin and hair) describes methods and compositions for increasing pigmentation in hair, preferably having methylthioadenosine.

Documents WO 2004/091558 A2 (Orlow et al., 2004, Compound for stimulating and for inhibiting melanin formation, and method for screening these compounds) describes a method for decreasing and/or increasing pigmentation of hair by administering an effective amount of at least one tri-substituted triazine compound.

Document US 7014844 B2 (Mahalingam et al., 2006, Lightening compositions and methods of use) describes the use of N,N,S (tris) carboxylmethylcysteamine in topical compositions as a lightening agent of hair.

Document WO 2008/155048 Al (Moser et al., 2008, Cosmetic compositions comprising sclareolide and hesperidin methyl chalcone) describes the use of cosmetic compositions comprising sclareolide and hesperidin methyl chalcone for the darkening of hair.

Document WO 2010/049463 Al (Philippe et al., 2010, Depigmenting topical compositions and their uses) describes the use of rucinol or one of its salts in a topical composition to whiten human non-scalp or scalp hair.

Documents US 2010/0323962 A1 and EP 2 489 363 A2 (Ramaiah, 2010 and 2012, Agonist peptides of basic fibroblast growth factor (BFGF) and the method of reduction of wrinkle on skin, darkening of hair and acceleration of wound healing) describes the use of a composition for the darkening of hair, comprising a peptide in combination with any known acceptable carrier for topical application.

Document US 8329149 B2 (Lyga et al., 2012, Topical lightening composition and uses thereof) describes a topical composition comprising substituted-4-oxobutanoic acid, ester, or amide tyrosinase inhibitor that are effective to lighten hair.

Document WO 2013/030794 A2 (Kasraee et al., 2013, Use of substituted pyridines as skin depigmenting compounds) describes hair depigmenting compositions comprising a pyridine derivative.

Document WO 2014/125452 A1 (Giuliani et al., 2014, Composition for cosmetic use suitable to produce a pigmentation effect on hair) describes topical cosmetic compositions based on spermidine designed to promote hair pigmentation.

Document US 9060949B2 (Vielhaber et al., 2015, Methyl carbamate compounds as skin and/or hair lightening actives) describes a composition for lightening human hair.

Document WO 2017/207428 Al (Kasraee et al., 2017, Use of thiophosphate derivatives as skin depigmenting agents) describes hair depigmenting compositions comprising at least one thiophosphate derivative.

In such patents, although in vitro (cell culture, enzymatic assays) and/or ex vivo (hair follicles culture, skin equivalents, etc.) data is used to support the melanogenic effect of such agents, an unequivocal in vivo proof of hair colour modulation is frequently neglected, only performed in animal models or related to abnormal hair pigment conditions that not always are directly related to melanin production. Topical compositions with proven effect in the darkening and/or lightening of human hair are still a need in the art.

A conspicuous effort to reduce the problems incidence related to the curling/waving of normally straight and the straightening of kinky hair can be found in the art. However, all proposed methods still rely in somehow inconvenient physical and harsh chemical treatments of hair fibres which are dangerous to the skin, eyes and hair at some degree. Since alternative treatments remain a need in the cosmetic industry, the present invention proposes a completely new approach to the modulation of hair shape which intends to provide the consumer with a great choice of options and products for a safe use at home.

These facts are disclosed in order to illustrate the technical problem addressed by the present disclosure.

### GENERAL DESCRIPTION

The present application discloses cosmetic formulations containing new bioactive agents, highly marketable, conceived to safely modulate hair colour and/or shape and/or volume from the follicle.

In the present disclosure, a modulation bioactive agent is a newly repurposed well-known drug that demonstrated in vitro ability to stimulate or inhibit the melanogenesis pathway or to modify the expression of a set of genes found to be differently active between curly and straight human hair follicles.

The cosmetic technology associated to their usage represents a change in the paradigm of cosmetic performance at the fibre level. In an embodiment, upon topical delivery to the scalp, these bioactive agents are capable of regulating the activity of target genes and/or proteins in cells of the hair follicle, therefore, changing as desired the hair phenotype as it is actively produced at the root and grows out of the skin.

This technology is unique regarding what is already protected and/or currently commercialized. In an embodiment, the hair volume, shape and colour changes are attained by modifying those attributes on the new hair that is being produced by the cells of the hair bulb under the influence of these bioactive agents - hair follicle modulation. Current hair technologies target the hair dead fibre, out of the skin or the scalp.

These agents can be used separately or in combination of two or more, provided that they induce the same intended alteration to the hair.

The bioactive agents can be included in any intended cosmetic formulation. They are suitable for topical application in the skin of a mammal, preferably the skin of the human scalp.

In an embodiment, the concentration of each bioactive agent to be used depends on their pharmacodynamics/pharmacokinetics properties, but also on the hair properties and type, and on the formulation.

In an embodiment, formulations containing the bioactive agents can be prepared by conventional methods. Additionally, many companies provide customized services regarding the preparation of formulations for scalp applications.

The formulation used in the disclosed examples was specifically tuned to target the hair follicle in order to efficiently deliver the active ingredients at the target site, the hair follicle cells.

Considering the positive results obtained with human volunteers in a clinical study at a pilot scale, the present disclosure has a Technology Readiness Level (TRL) of 5-6.

The use of the composition described in this application do not comprise the disadvantages of other technologies found in the state of the art in the field. The composition is of easy application, do not compromise the properties (as mechanical strength) of the hair fibre and do not induce scalp irritation or other skin health issue.

The technology related to the present disclosure provides a significantly positive impact in the hair cosmetics industry by contributing with safer, greener, innovative and targeted cosmetics, which is able to interfere with melanin production and fibre wave formation at the hair follicle.

The present disclosure relates to a composition for use in the hair follicle modulation comprising a bioactive agent selected from the following list: dipyridamole, dipyridamole salt, rivastigmine, rivastigmine salt, paroxetine, paroxetine salt, ethacrynic acid, ethacrynic salt, midodrine, midodrine salt, topiramate, topiramate salt, entacapone, entacapone salt, or mixtures thereof; a thickener agent; a preservative; and a denaturing alcohol.

The present disclosure also relates to a composition for hair follicle modulation comprising: a bioactive agent selected from the following list: dipyridamole, dipyridamole salt, rivastigmine, rivastigmine salt, paroxetine, paroxetine salt, ethacrynic acid, ethacrynic salt, midodrine, midodrine salt, topiramate, topiramate salt, entacapone, entacapone salt, or mixtures thereof; a thickener agent; a preservative; and a denaturing alcohol; wherein hair follicle modulation is a hair colour modulation and/or a hair volume modulation; wherein paroxetine, and/or paroxetine salt are not use for a hair volume modulation.

In an embodiment, the bioactive agent is selected from the following list: dipyridamole, dipyridamole salt, rivastigmine, rivastigmine salt, ethacrynic acid, ethacrynic salt, midodrine, midodrine salt, topiramate, topiramate salt, entacapone, entacapone salt, or mixtures thereof.

In an embodiment, the composition comprises 0.001-50% by weight of the bioactive agent with respect to the total weight of the composition, preferably 0.01-25% by weight of the bioactive agent, more preferably 0.2-8% by weight of the bioactive agent.

In an embodiment, the thickener agent is selected from a list of: sodium acrylates copolymer and lecithin; aluminum stearates/isostearates/myristates/laurates/palmitates, glycol distearate, hydrogenated castor oil, hydrogenated castor oil hydroxystearate, hydrogenated castor oil isostearate, hydrogenated castor oil stearate, hydrogenated castor PEG-8 esters, PEG-150 distearate, polyethylene glycol, polyacrylic acid/carbomer, carcomer 934, tea-carbomer, carboxymethyl chitin, carboxymethyl chitosan, carboxymethyl dextran, carboxymethyl hydroxypropyl guar, sodium carbomer, sodium dextran sulfate, sodium polystyrene sulfonate, sodium surfactin, stearalkonium bentonite, stearalkonium hectorite, steareth-30/-40/-50, xanthan gum, vegetable gums, Ahnfeltia confina extract, Euglena gracilis polysaccharide or mixtures thereof.

In an embodiment, the preservative agent is selected from a list consisting of: phenoxyethanol and ethylhexylglycerin; butyl paraben, diazolidinyl urea, DMDM hydantoin, ethyl paraben, imidazolidinyl urea, iodopropynyl butylcarbamate, isobutyl paraben, methyl paraben, methylchloroisothiazolinone, methylisothiazolinone, phenoxyethanol, ethylhexylglycerin, propyl paraben, sodium benzoate, Germaben II, Germall Plus, Kathon, potassium sorbate/ sorbic acid, quaternium-15, polyoxymethylene urea, sodium hydroxymethylglycinate, bromopol, glyoxal, isopropylparaben, benzylate, benzoic acid, and benzyl ester, triclosan and triclocarban, benzyl alcohol, benzalkonium chloride, citric acid, dehydroacetic acid, essential oils, grapefruit seed extract, lactic acid, levulinic acid, sodium dehydroacetate, sodium metabisulfite, sodium salicylate, vitamin E, zinc pyrithione, or mixtures thereof.

In an embodiment, the denaturing alcohol is selected from a list of diethylene glycol monoethyl ether; 1,2,6 hexanetriol, dipropylene glycol, glycerin, hexylene glycol, panthenol, phytantriol, propylene glycol, sodium pyroglutamic acid (PCA), sorbitol, diethylene glycol monoethyl ether, triethylene glycol, polyglyceryl sorbitol, glucose, fructose, polydextrose, potassium PCA, hydrogenated honey, hyaluronic acid, inositol, hexanediol beeswax, hexanetriol beeswax, hydrolyzed elastin, hydrolyzed collagen, hydrolyzed silk, hydrolyzed keratin, erythritol, capryl glycol, isoceteth-(3-10, 20, 30), isolaureth-(3-10, 20, 30), laneth-(5-50), laureth-(1-30), steareth-(4-20), trideceth-(5-50), lithium chloride, glyceryl triacetate, butylene glycol, lactic acid, aloe vera, xylitol, maltitol, castor oil, urea, tripropylene glycol, collagen, pentylene glycol, or mixtures thereof.

In an embodiment, the hair follicle modulation is a hair colour modulation and/or a hair volume modulation. In another embodiment, the hair colour modulation comprises hair darkening or hair lightening. In a yet another embodiment, the hair volume modulation comprises hair straightening or hair curling.

In an embodiment, the colour modulation is regulated by dipyridamole, dipyridamole salt, rivastigmine, rivastigmine salt, paroxetine, paroxetine salt or mixtures thereof. In another embodiment, the hair darkening is regulated by dipyridamole or dipyridamole salt. In yet another embodiment, the hair lightening is regulated by rivastigmine, rivastigmine salt, paroxetine, paroxetine salt or mixtures thereof.

In an embodiment, the hair volume modulation is regulated by ethacrynic acid, ethacrynic salt, midodrine, midodrine salt, topiramate, topiramate salt, entacapone, entacapone salt, or mixtures thereof. In a further embodiment, hair straightening is regulated by ethacrynic acid, ethacrynic salt, midodrine, midodrine salt, or mixtures thereof. In another embodiment, hair curling is regulated by topiramate, topiramate salt, entacapone, entacapone salt, or mixtures thereof.

In an embodiment, hair modulation comprises hair darkening and hair straightening. In another embodiment, hair modulation comprises hair darkening and hair curling. In yet another embodiment, hair modulation comprises hair lightening and hair straightening. In a further embodiment, hair modulation comprises hair lightening and hair curling.

In an embodiment, the bioactive agent is encapsulated or bonded to a controlled release system, in particular a liposome.

In an embodiment, the composition is a topical composition. In particular, the composition related to the present disclosure is administered topically to the skin of a mammal, preferably the skin of the human scalp.

In an embodiment, the topical composition is a solution, hydroalcoholic solution, dispersion, suspension, tonic, emulsion, lotion, elixir, serum, toner, cleanser, cream, mask, mousse, ointment, gel, wax, oil, foam, soap, shampoo, conditioner, spray, aerosol, powder, paste.

In another embodiment, the composition further comprises at least one cosmetically and/or pharmaceutically and/or dermatologically acceptable excipient.

In an embodiment, the composition further comprises at least one of the excipients and/or compounds selected from the following list: surfactants, anionic surfactant, amphoteric surfactant, cationic surfactant, non-ionic surfactant emulsifiers, preservatives, thickeners, natural polymers derivative, organic polymers, proteins, humectants, cationic polymers, silicones, oils (including organic and natural oils), fragrances, vitamins, emollient esters, alkanolamides, amines, buffers, pH adjustors, salts, antimicrobial agents, antibacterial agents, aliphatic alcohols, UV filters/sunscreens, amine oxides, chelates, fatty acids, PEG-modified materials, polymers, anti-static agents, alcohols, disinfectants agents, or any mixture thereof.

In an embodiment, the composition described in the present disclosure is for use in medicine, veterinary or cosmetics; in particular for use in the prevention, therapy or treatment of abnormal defective production of melanin. In a further embodiment, the present disclosure relates to a composition for use as self-tanner agent.

The present disclosure also relates to a pharmaceutical composition for use in the prevention, therapy or treatment of abnormal defective production of melanin, comprising a bioactive agent selected from the following list: dipyridamole, dipyridamole salt, rivastigmine, rivastigmine salt, paroxetine, paroxetine salt, or mixtures thereof. In an embodiment, the bioactive agent is encapsulated or bonded to a controlled release system, in particular a liposome.

In an embodiment, the pharmaceutical composition can be used in the prevention, therapy or treatment of melasma; solar or senile lentigo, freckles due to abnormal excessive production of melanin, post-medicinal hyperpigmentation, post-inflammatory hyperpigmentation, light induced hyperpigmentation and chemical induced hyperpigmentation.

In an embodiment, the pharmaceutical composition may further comprise a pharmaceutical acceptable thickener agent; a pharmaceutical acceptable preservative; and a pharmaceutical acceptable denaturing alcohol.

An aspect of the present disclosure comprises the use of a bioactive agent as a hair modulator wherein the bioactive agent is selected from the following list: dipyridamole, dipyridamole salt, rivastigmine, rivastigmine salt, paroxetine, paroxetine salt, ethacrynic acid, ethacrynic salt, midodrine, midodrine salt, topiramate, topiramate salt, entacapone, entacapone salt, or mixtures thereof. In an embodiment, the bioactive agent is encapsulated or bonded to a controlled release system, in particular a liposome.

In an embodiment, the hair modulation is a colour modulation and/or a volume modulation. In particular, the hair colour modulation comprises darkening or lightening of the hair fibre, and the hair volume modulation comprises hair straightening or hair curling.

In an embodiment, the bioactive agent can be used as a hair modulator in hair follicle modulation, wherein the bioactive agent is selected from the following list: dipyridamole, dipyridamole salt, rivastigmine, rivastigmine salt, paroxetine, paroxetine salt, ethacrynic acid, ethacrynic salt, midodrine, midodrine salt, topiramate, topiramate salt, entacapone, entacapone salt, or mixtures thereof, wherein the hair modulator is a hair colour modulator and/or a hair volume modulator; wherein paroxetine, and/or paroxetine salt are not use as hair volume modulator.

In an embodiment, the bioactive agent can be used as hair colour modulator and/or hair volume modulator.

In a further embodiment, the bioactive agent can be used as hair colour modulator, wherein the colour modulator is a darkener or lightener of the hair fibre. In a yet further embodiment, the bioactive agent can be used as hair volume modulator, wherein the volume modulator is a hair straightener or hair curler.

The present disclosure also relates to a kit, cosmetic product or reagent comprising the bioactive agent as described in the present disclosure.

### DETAILED DESCRIPTION

The present disclosure proposes a composition for the hair follicle modulation, comprising a bioactive agent, a thickener, a preservative and a denaturing alcohol. This new concept to change hair morphology consists in the topical delivery of compounds able to regulate the activity of target genes and/or proteins in cells of the hair follicle, modelling the hair phenotype as it is actively produced in the bulb and grows out of the skin. All the available methods to change colour and shape act externally on the hair fibre and are based on cosmetic emulsions with alkaline pH and/or strong redox power with very negative consequences for hair, scalp hair, skin, scalp and even to consumers’ health and environment.

In an embodiment, the hair colour modulation is regulated by dipyridamole, dipyridamole salt, rivastigmine, rivastigmine salt, paroxetine, paroxetine salt or mixtures thereof. In a further embodiment, the change in hair colour consists in the topical delivery of dipyridamole and/or one of its pharmacologically acceptable salts to promote the darkening of hair. In a yet further embodiment, the change in hair colour consists in the topical delivery of rivastigmine and/or paroxetine and/or one of their pharmacologically acceptable salts to promote the lightening of hair.

In an embodiment, the hair shape/volume is regulated by ethacrynic acid, ethacrynic salt, midodrine, midodrine salt, topiramate, topiramate salt, entacapone, entacapone salt, or mixtures thereof. In a further embodiment, the change in hair shape consists in the topical delivery of ethacrynic acid and/or midodrine to promote the straightening/volume reduction of hair. In a yet further embodiment, the change in hair shape consists in the topical delivery of topiramate and/or entacapone to promote and/or to increase the curling/volume of hair.

In yet another embodiment, said topical delivery implies the application of the compounds to the skin of a mammal, preferably the skin of the human scalp.

In an embodiment, the continuous use of the composition containing at least one of the bioactive agents described in the present disclosure, provides hair shape and/or colour change.

The concentration of each modulation bioactive agents to be used depends on their pharmacodynamics/pharmacokinetics properties but also on the hair properties and type.

In an embodiment, the concentration of modulation bioactive agents in a composition, namely Dipyridamole, varies from 0.001% to 35% (weight by weight), preferentially from 0.01% to 3.5%.

In an embodiment, the concentration of modulation bioactive agents in a composition, namely Rivastigmine, varies from 0.001% to 20% (weight by weight), preferentially from 0.2% to 2%.

In an embodiment, the concentration of modulation bioactive agents in a composition, namely Paroxetine, varies from 0.001% to 30% (weight by weight), preferentially from 0.3% to 3%.

In an embodiment, the concentration of modulation bioactive agents in a composition, namely Topiramate, varies from 0.001% to 40% (weight by weight), preferentially from 0.2% to 20%.

In an embodiment, the concentration of modulation bioactive agents in a composition, namely Entacapone, varies from 0.001% to 50% (weight by weight), preferentially from 0.01% to 25%.

In an embodiment, the concentration of modulation bioactive agents in a composition, namely Midodrine, varies from 0.001% to 25% (by weight), preferentially from 0.25% to 2.5%.

In an embodiment, the concentration of modulation bioactive agents in a composition, namely Ethacrynic Acid, varies from 0.001% to 30% (by weight), preferentially from 0.8% to 8%.

In an embodiment, the composition for use in the hair follicle modulation can comprise at least one excipient, selected from the following list: surfactants, anionic surfactant, amphoteric surfactant, cationic surfactant, non-ionic surfactant, emulsifiers, preservatives, thickeners, natural polymers derivatives, organic polymers, proteins, humectants, silicones, oils (including organic oils), fragrances, vitamins, emollient esters, alkanolamides, amines, buffers, pH adjustors, salts, antimicrobial agents, antibacterial agents, aliphatic alcohols, UV filters, amine oxides, chelates, fatty acids, polyethylene glycol (PEG) materials, polymers, anti-static agents, alcohols, disinfectants agents, or any mixture thereof.

In other embodiment, the composition with modulation bioactive agents for scalp application can comprise at least one anionic surfactant selected from the following list: alkylbenzene sulfonates, ammonium lauryl sulfate, ammonium lauryl sulfate, ammonium xylenesulfonate, sodium C14-16 olefin sulfonate, sodium cocoyl sarcosinate, sodium laureth sulfate, sodium lauryl sulfate, sodium lauryl sulfoacetate, sodium myreth sulfate, sodium xylenesulfonate, TEA dodecylbenzenesulfonate, ethyl PEG-15 cocamine sulfate, dioctyl sodium sulfosuccinate, or any mixture thereof.

In an embodiment, the composition can comprise at least one amphoteric surfactant selected from the following list: cocamidopropyl betaine, coco betaine, cocoamphoacetate, cocoamphodipropionate, disodium cocoamphodiacetate, disodium cocoamphodipropionate, lauroamphoacetate, sodium cocoyl isethionate, or any mixture thereof.

In other embodiment, the composition can comprise at least one cationic surfactant selected from the following list: quaternary ammonium compounds, behentrimonium chloride, behentrimonium methosulfate, benzalkonium chloride, betrimonium chloride, binnamidopropyltrimonium chloride, cocotrimonium chloride, dicetyldimonium chloride, dicocodimonium chloride, dihydrogenated tallow dimethylammonium chloride, hydrogenated Palm trimethylammonium chloride, laurtrimonium chloride, quaternium-15, quaternium-18 bentonite, quaternium-22 hectonite, stearalkonium chloride, tallowtrimonium chloride, tricetyldimonium chloride, or any mixture thereof.

In yet other embodiment, the composition can comprise at least one non-ionic surfactant selected from the following list: decyl glucoside, laureth-10 (lauryl ether 10), laureth-23, Laureth-4, PEG-10 sorbitan laurate, polysorbate-(20, 21, 40, 60, 61, 65, 80, 81), PPG-1 trideceth-6, sorbitol, steareth-(2, 10, 15, 20), C11-21 pareth-(3-30), C12-20 acid PEG-8 ester, or their mixtures.

In yet other embodiment, the composition can comprise at least one emulsifier selected from the following list: phosphatidylcholine, caprylic/capric/diglyceryl succinate, C10-15 pareth-(2,4,6,8) phosphate, C14-16 glycol palmitate, C18-20 glycol isostearate, ceteareth-(4-60), cocamidopropyl lauryl ether, deceth-(3-10), DIPA-hydrogenated cocoate, dipentaerythrityl hydroxystearate, dipentaerythrityl hydroxyisostearate, dipentaerythrityl hexacaprate/caprylate, dodoxynol-( 5,6,7,9,12), nonoxynol-(1-35), octoxynol-(1 70), Octyldodeceth-(2,5,16,20,25), Palm kernel glycerides, or any mixture thereof.

In other embodiment, the composition can comprise at least one preservative selected from the following list: butyl paraben, diazolidinyl urea, DMDM hydantoin, ethyl paraben, imidazolidinyl urea, iodopropynyl butylcarbamate, isobutyl paraben, methyl paraben, methylchloroisothiazolinone, methylisothiazolinone, phenoxyethanol, ethylhexylglycerin, propyl paraben, sodium benzoate, or any mixture thereof.

In other embodiment, the composition can comprise at least one thickener selected from the following list: sodium acrylates copolymer, aluminium stearates/isostearates/myristates/laurates/palmitates, glycol distearate, hydrogenated castor oil, hydrogenated castor oil hydroxystearate, hydrogenated castor oil isostearate, hydrogenated castor oil stearate, hydrogenated castor PEG-8 esters, PEG-150 distearate, polyethylene glycol, polyacrylic acid/carbomer, carcomer 934, tea-carbomer, carboxymethyl chitin, carboxymethyl chitosan, carboxymethyl dextran, carboxymethyl hydroxypropyl guar, sodium carbomer, sodium dextran sulfate, sodium polystyrene sulfonate, sodium surfactin, stearalkonium bentonite, stearalkonium hectorite, steareth-30/-40/-50, xanthan gum, vegetable gums, Ahnfeltia confina extract, Euglena gracilis polysaccharide or any mixture thereof.

In other embodiment, the composition can comprise at least one natural polymer derivative selected from the following list: carboxymethyl hydroxyethyl cellulose, carboxymethyl hydroxypropyl guar, cellulose, ethyl cellulose, hydroxybutyl methylcellulose, hydroxyethylcellulose, hydroxymethylcellulose, lauryl polyglucose, or any mixture thereof.

In other embodiment, the composition can comprise at least one humectant selected from the following list: 1,2,6 hexanetriol, dipropylene glycol, glycerin, hexylene glycol, panthenol, phytantriol, propylene glycol, sodium pyroglutamic acid (PCA), sorbitol, diethylene glycol monoethyl ether, triethylene glycol, polyglyceryl sorbitol, glucose, fructose, polydextrose, potassium PCA, hydrogenated honey, hyaluronic acid, inositol, hexanediol beeswax, hexanetriol beeswax, hydrolyzed elastin, hydrolyzed collagen, hydrolyzed silk, hydrolyzed keratin, erythritol, capryl glycol, isoceteth-(3-10, 20, 30), isolaureth-(3-10, 20, 30), laneth-(5-50), laureth-(1-30), steareth-(4-20), trideceth-(5-50), or any mixture thereof.

In other embodiment, the composition can comprise at least one cationic polymer selected from the following list: polyquaternium-10, polyquaternium-7, polyquaternium-11m guar hydroxypropyltrimonium chloride, or any mixture thereof.

In other embodiment, the composition can comprise at least one silicone selected from the following list: amodimethicone, amodimethicone, trideceth-12, cetrimonium, chloride mixture, behenoxy, dimethicone sparingly, cetearyl methicone, cetyl dimethicone, cyclomethicone, cyclopentasiloxane, dimethicone, dimethicone copolyol, dimethicone copolyol, dimethiconol, hydrolyzed wheat protein hydroxypropyl polysiloxane, stearoxy dimethicone sparingly, stearyl dimethicone, trimethylsilylamodimethicone, lauryl methicone copolyol, or any mixture thereof.

In yet other embodiment, the composition comprise at least one organic oil selected from the following list: mineral oil, paraffin, petrolatum, or any mixture thereof.

In yet other embodiment, the composition can comprise at least one protein selected from the following list: cocodimonium hydroxypropyl hydrolyzed casein, cocodimonium hydroxypropyl hydrolyzed collagen, cocodimonium hydroxypropyl hydrolyzed hair keratin, cocodimonium hydroxypropyl hydrolyzed keratin, cocodimonium hydroxypropyl hydrolyzed rice protein, cocodimonium hydroxypropyl hydrolyzed silk, cocodimonium hydroxypropyl hydrolyzed soy protein, cocodimonium hydroxypropyl hydrolyzed wheat protein, cocodimonium hydroxypropyl silk amino acids, cocoyl hydrolyzed collagen, cocoyl hydrolyzed keratin, hydrolyzed keratin, hydrolyzed oat flour, hydrolyzed silk, hydrolyzed silk protein, hydrolyzed soy protein, hydrolyzed wheat protein, hydrolyzed wheat protein, keratin, potassium cocoyl hydrolyzed collagen, TEA-cocoyl hydrolyzed collagen, TEA-cocoyl hydrolyzed soy protein, or any mixture thereof.

In other embodiment, the composition can comprise at least one vitamin selected from the following list: retinol, retinyl palmitate tocopherol acetate, or any mixture thereof.

In other embodiment, the composition can comprise at least one emollient ester selected from the following list: butyl myristate, butyl stearate, C12-15 alkyl benzoate, caprylic/capric triglyceride, cetyl octanoate, cetyl stearate, cetearyl stearate, decyl oleate, dimethyl lauramine isostearate, glyceryl stearate, glyceryl adipate, glyceryl arachidate, glyceryl arachidonate, glyceryl behenate, glyceryl caprate, glyceryl caprylate, glyceryl caprylate/ caprate, glyceryl citrate/lactate/linoleate/oleate, glyceryl cocoate, glyceryl diarachidate, glyceryl dibehenate, glyceryl dierucate, glyceryl dihydroxystearate, glyceryl diisopalmitate, glyceryl diisostearate, glyceryl dilaurate, glyceryl dilinoleate, glyceryl dimyristate, glyceryl dioleate, glyceryl dipalmitate, glyceryl dipalmitoleate, glyceryl diricinoleate, glyceryl distearate, glyceryl erucate, glycol stearate, isocetyl stearate, isopropyl myristate, isopropyl palmitate, isopropyl stearate, isostearyl stearate, octyl palmitate, octyl stearate, propylene glycol dicaprylate/dicaprate, sorbitan benzoate, sorbitan caprylate, sorbitan isostearate, sorbitan laurate, sorbitan tristearate, stearyl stearate, tocopheryl linoleate, or any mixture thereof.

In other embodiment, the composition can comprise at least one alkanolamide selected from the following list: acetamide MEA (monoethanolamine), cocamide DEA (diethanolamine), cocamide MEA, lactamide MEA, lauramide DEA, propylene glycol, lauramide MEA, lecithinamide DEA, linoleamide DEA, linoleamide MEA, linoleamide MIPA, myristamide DEA, myristamide MEA, myristamide MIPA, oleamide DEA, oleamide DEA, oleamide MEA, oleamide MIPA, soyamide DEA, stearamide MEA, or any mixture thereof.

In yet other embodiment, the composition can comprise at least one amine selected from the following list: behentamidopropyl dimethylamine, cocamidopropyl dimethylamine, isostearamidopropyl dimethylamine, lauramidopropyl dimethylamine, myristamidopropyl dimethylamine, oleamidopropyl dimethylamine, palmitamidopropyl dimethylamine, stearamidopropyl dimethylamine, tallamidopropyl dimethylamine, or any mixture thereof.

In yet other embodiment, the composition can comprise at least one pH adjuster selected from the following list: ascorbic acid, citric acid, sodium hydroxide, triethanolamine, or any mixture thereof.

In yet other embodiment, the composition can comprise at least one salt selected from the following list: calcium chloride, magnesium chloride, magnesium sulfate, potassium chloride, potassium glycol sulfate, sodium chloride, or any mixture thereof.

In yet another embodiment, the composition can comprise at least one aliphatic alcohol selected from the following list: ethanol, behenyl alcohol, cetearyl alcohol, cetyl alcohol, isocetyl alcohol, isostearyl alcohol, lauryl alcohol, myristyl alcohol, stearyl alcohol, C30-50 alcohols, lanolin alcohol, or any mixture thereof.

In another embodiment, the composition can comprise at least one UV filter/sunscreen selected from the following list: benzophenone-(2, 3, 4, 5, 6, 7, 8, 9, or 10), benzophenone-4, benzyl salicylate, benzylidene camphor sulfonic acid, bornelone, ethyl cinnamate, ethylhexyl methoxycinnamate (octyl methoxycinnamate), octoxynol-40, octoxynol-20, octyl methoxycinnamate, octyl salicylate, oxybenzone, phenyl ketone, PEG-25 PABA, polyacrylamidomethyl benzylidene camphor, or any mixture thereof.

In another embodiment, the composition can comprise at least one natural oil selected from the following list: coconut oil, jojoba oil, olive oil, palm Oil, safflower oil, sesame seed oil, shea butter, sweet almond oil, wheat germ oil, or any mixture thereof.

In yet other embodiment, the composition can comprise at least one amine oxide selected from the following list: cocamine oxide, lauramine oxide, or any mixture thereof.

In another embodiment, the composition can comprise at least one chelate selected from the following list: diiospropyl oxalate, disodium EDTA (ethylenediaminetetraacetic acid), disodium EDTA-copper, HEDTA (hydroxyethyl ethylenediamine triacetic acid), oxalic acid, potassium citrate, sodium citrate, dodium oxalate, TEA-EDTA (triethanolamine-EDTA), tetrasodium EDTA, trisodium EDTA, trisodium HEDTA, or any mixture thereof.

In another embodiment, the composition can comprise at least one fatty acid selected from the following list: arichidonic acid, capric acid, coconut fatty acid, lauric acid, linoleic acid, linolenic acid, myristic acid, palmitic acid, pantothenic acid, stearic acid, caproic acid, capryleth-(4, 6, 9) carboxylic acid, isostearic acid, or any mixture thereof.

In another embodiment, the composition can comprise at least one antimicrobial/antibacterial agent selected from the following list: glyoxal, triclosan, or any mixture thereof.

In another embodiment, the composition can comprise at least one PEG-modified material selected from the following list: PEG-150 pentaerythirtyl tetrastearate, PEG-(-2, -3, -4, -6, -8, -12, -20, -32, -50, -150, -175) distearate, PEG-10 castor oil, PEG-10 cocamine, PEG-10 cocoate, PEG-10 coconut oil esters, PEG-10 glyceryl oleate, PEG-10 glyceryl pibsa tallate, PEG-10 glyceryl stearate, PEG-10 hydrogenated lanolin, PEG-10 hydrogenated tallow amine, PEG-10 isolauryl thioether, PEG-10 isostearate, PEG-10 lanolate, PEG-10 lanolin, PEG-10 laurate, PEG-10 oleate, PEG-10 olive glycerides, PEG-10 polyglyceryl-2 laurate, PEG-10 propylene glycol, PEG-10 sorbitan laurate, PEG-10 soya sterol, PEG-10 soyamine, PEG-10 stearamine, PEG-10 stearate, PEG-10 stearyl benzonium chloride, PEG-10 tallate, PEG-10 tallow aminopropylamine, PEG-100, PEG-100 castor oil, PEG-100 hydrogenated castor oil, PEG-100 lanolin, PEG-100 stearate, PEG-40 hydrogenated castor Oil, PEG 60, PEG-55 propylene glycol distearate, or any mixture thereof.

In another embodiment, the composition can comprise at least one polymer selected from the following list: carbomer, dodecanedioic acid/cetearyl alcohol/glycol copolymer, hydrogenated C6-14 olefin polymers, hydrogenated ethylene/propylene/styrene copolymer: polyacrylic acid, polymethyl methacrylate: polymer, polyvinyl acetate, polyvinyl alcohol, polypropylene glycol (PPG), PPG-25-laureth-25, PPG-5 pentaerithrityl ether, PPG-75-PEG-300-hexylene glycol, polyvinylpyrrolidone, PVP/VA (polyvinylpyrrolidone/vinyl acetate copolymer), sodium carbomer, TEA-carbomer, poloxamer (100-407), poloxamine, polyacrylamidomethylpropane sulfonic acid, polyethylene terephthalate, or any mixture thereof.

In another embodiment, the composition can comprise at least one antistatic agent selected from the following list: apricotamidopropyl ethyldimonium ethosulfate, apricotamidopropyl ethyldimonium lactate, cocamidopropyl ethyldimonium ethosulfate, cocamidopropyl ethyldimonium lactate, lauramidopropyl ethyldimonium ethosulfate, lauramidopropyl ethyldimonium lactate, linoleamidopropyl ethyldimonium ethosulfate, linoleamidopropyl ethyldimonium lactate, myristamidopropyl ethyldimonium ethosulfate, myristamidopropyl ethyldimonium lactate, oleamidopropyl ethyldimonium ethosulfate, oleamidopropyl ethyldimonium lactate, stearamidopropyl ethyldimonium ethosulfate, stearamidopropyl ethyldimonium lactate, or any mixture thereof.

In another embodiment, the composition can comprise at least one alcohol selected from the following list: SD alcohol 40, isopropanol, or any mixture thereof.

In yet another embodiment, the composition for use in the hair follicle modulation can comprise further beneficial agents for the skin and/or hair.

In an embodiment, the composition for use in the hair follicle modulation is a topical composition, that can be in the form of, but not limited to, solution, hydroalcoholic solution, dispersion, suspension, shampoo, lotion, serum, tonic, emulsion, toner, cleanser, cream, mousse, ointment, gel, wax, conditioner, foam, elixir, oil, spray, aerosol, soap, powder, skin patch or mask.

In another aspect of the previous embodiment, regardless of the form of the compositions, they can contain bioactive agents liposomated, complexed or in any controlled release system.

The present disclosure describes the composition of a solution that helps the active ingredients to permeate the skin and diffuse into hair follicles, changing the properties of the hair, as a preferred embodiment of a composition.

In another embodiment, formulations with modulation bioactive agents for scalp application can be used in medicine, veterinary and/or cosmetics, said usage comprising application to the skin of a mammal, especially the skin of the human scalp, for straightening the hair, curling the hair, darkening the hair, lightening the hair or as a volumizing agent.

In another embodiment, formulations with dipyridamole and/or paroxetine hydrochloride and/or one of their pharmacologically acceptable salts can be used in medicine, veterinary and/or cosmetics, said usage comprising application to the skin of a mammal, especially the human skin, to modulate the production of melanin in the skin.

### EXAMPLES OF APPLICATIONS

The examples are within the scope of the claims and represent different embodiments of the invention.

### Example 1

This example discloses an “in vitro” treatment of human melanocytes (SK-MEL-23 cell line) with a solution containing a modulation bioactive agent for darkening the skin.

In an embodiment, the bioactive agent used in this example was Dipyridamole as darkening agent in a solution containing water and DMSO.

Cells were seeded on 24-well plates at density of 9.0x10⁴ cells/well and treated next day with 10 µM of dipyridamole and 1% (v/v) DMSO. In the control, cells were seeded on 24-well plates at density of 9.0x10⁴ cells/well and treated next day with 1% (v/v) DMSO.

The intracellular melanin quantification was performed after 3 days. The method for quantifying melanin in hair was as described by Fernandes B. et al., 2016 [51]. The melanin contents were normalized by protein levels in each sample (DC Protein assay). Melanin change in cells treated with Dipyridamole was calculated using the following mathematical formula:

$$\begin{matrix}E f f e c t o n m e l a n i n \left(\%\right) = \\ \frac{N o r m a l i z e d m e l a n i n c o n t e n t o f t r e a t e d c e l l s}{n o r m a l i z e d m e l a n i n c o n t e n t o f v e h i c l e c o n t r o l} \times 100\end{matrix}$$

The treatment of melanocytes with the modulation bioactive agent Dipyridamole induced an increase in melanin production by 3.5-fold relative to the control experiment.

All the results present on the following described examples were performed in a pilot clinical study with intervention of cosmetic products (RNEC number: 92938), not contested by the Portuguese Legal Competent Authorities.

### Example 2

This example discloses the treatment of human scalp with a formulation containing a modulation bioactive agent for straightening the hair.

The bioactive agent used in this example was Midodrine hydrochloride as straightening agent in a formulation containing water, a thickener agent with emulsifying properties - Lecigel™, a preservative agent – Euxyl® PE 9010, denaturing alcohol and Transcutol® CG.

The volunteers for the application test of the formulation with the modulation bioactive agent Midodrine hydrochloride had curly hair.

Each volunteer received the formulation without the agent Midodrine hydrochloride and the formulation with the agent Midodrine hydrochloride. The formulation that contained the modulation bioactive agent had a concentration of 0.25% (weight by weight) Midodrine hydrochloride.

On each volunteer, the hair was shaved in two different posterior scalp locations, of approximately 1 cm² each, and the formulations were applied for 5 weeks, three times a week.

The formulations with and without Midodrine were applied directly on scalp. Ten microliters per 1 cm² of the control formulation without Midodrine hydrochloride were applied to one of the two shaved scalp areas while ten microliters per 1 cm² of the formulation with Midodrine hydrochloride were applied to the other shaved scalp area.

After the 15^{th} application of formulations with and without the modulation agent, a final picture was taken showing both sites of application, the growth rate of the hairs was measured, and some hairs were plucked for further tests.

The curvature index was determined after the clinical cosmetic study conclusion, using hairs collected from the scalp areas treated with and without the modulation bioactive agent in a formulation. The control was the hair collected from scalp area treated with the formulation without the modulation bioactive ingredient.

The effect on curvature index was calculated according to the following formula:

$$\begin{matrix}\text{Effect} \text{on} \text{curvature} \text{index} \left(\text{\%}\right) \text{=} \\ \frac{\frac{r e l a x e d \overline{l e n g t h}}{s t r e s s e d \overline{l e n g t h}} \left(\text{control} \text{hair}\right)}{\frac{r e l a x e d \overline{l e n g t h}}{s t r e s s e d \overline{l e n g t h}} \left(\text{treated} \text{hair}\right)} \times 100 - 100\end{matrix}$$

Each hair was individually measured over a white paper (hair in a relaxed form) and a force was applied in order to keep the hair straight (under stress) and, in this way, measure the length of hair in stressed form.

The treatment of scalp with the modulation agent Midodrine hydrochloride induced a straightening effect. The hair that was very curly became visibly straighter, after just 5 weeks of treatment (Table 2).

### Example 3

This example discloses the treatment of human scalp with a formulation containing a modulation bioactive agent for curling the hair.

The modulation bioactive agent used in this example was Topiramate as curling agent in a formulation containing water, a thickener agent with emulsifying properties - Lecigel™, a preservative agent - Euxyl® PE 9010, denaturing alcohol and Transcutol® CG.

The volunteers for the application test of the formulation with the modulation agent Topiramate had straight hair.

Each volunteer received the formulation without the agent Topiramate and the formulation with the agent Topiramate. The formulation that contains the modulation bioactive agent had a concentration of 1.75% (weight by weight) Topiramate.

On each volunteer, the hair was shaved in two different posterior scalp locations, of approximately 1 cm² each, and the formulations were applied for 5 weeks, three times a week.

The formulations with and without Topiramate were applied directly on scalp. Ten microliters per 1 cm² of the control formulation without Topiramate were applied to one of the two shaved scalp areas while ten microliters per 1 cm² of the formulation with Topiramate were applied to the other shaved scalp area.

After the 15^{th} application of formulations with and without the modulation bioactive agent, a final picture was taken, the growth rate of the hairs was measured, and some hairs were plucked for further tests.

The curvature index was determined after the clinical cosmetic study conclusion, using hairs collected from the scalp areas treated with and without the modulation bioactive agent in a formulation. The control was the hair collected from the scalp area treated with the formulation without the modulation bioactive ingredient.

The effect on curvature index was calculated according to the following formula:

$$\begin{matrix}\text{Effect} \text{on} \text{curvature} \text{index} \left(\text{\%}\right) \text{=} \\ \frac{\frac{r e l a x e d \overline{l e n g t h}}{s t r e s s e d \overline{l e n g t h}} \left(\text{control} \text{hair}\right)}{\frac{r e l a x e d \overline{l e n g t h}}{s t r e s s e d \overline{l e n g t h}} \left(\text{treated} \text{hair}\right)} \times 100 - 100\end{matrix}$$

Each hair was individually measured over a white paper (hair in a relaxed form) and a force was applied in order to keep the hair straight (under stress) and, in this way, measure the length of hair in stressed form.

The treatment of scalp with the modulation agent Topiramate induced a curling effect. The hair that was straight became visibly more curved, after just 5 weeks of treatment (Table 2).

### Example 4

This example discloses the treatment of human scalp with a formulation containing a modulation bioactive agent for lightening the hair.

The modulation bioactive agent used in this example was Rivastigmine hydrogen tartrate as lightening agent in a formulation containing water, a thickener agent with emulsifying properties - Lecigel™, a preservative agent - Euxyl® PE 9010, denaturing alcohol and Transcutol® CG.

The volunteers for the application test of the formulation with the modulation agent Rivastigmine hydrogen tartrate had black to medium brown hair.

Each volunteer received the formulation without the agent Rivastigmine hydrogen tartrate and the formulation with the agent Rivastigmine hydrogen tartrate. The formulation that contains the modulation bioactive agent had a concentration of 0.2% (weight by weight) Rivastigmine.

On each volunteer, the hair was shaved in two different posterior scalp locations, of approximately 1 cm² each, and the formulations were applied for 5 weeks, three times a week.

The formulations with and without Rivastigmine were applied directly on scalp. Ten microliters per 1 cm² of the control formulation without Rivastigmine hydrogen tartrate were applied to one of the two shaved scalp areas while ten microliters per 1 cm² of the formulation with Rivastigmine hydrogen tartrate were applied to the other shaved scalp area.

After the 15^{th} application of formulations with and without the modulation agent, a final picture was taken, the growth rate of the hairs was measured, and some hairs were plucked for further tests.

The hair melanin quantification process was performed after the clinical cosmetic study conclusion, using hairs collected from the scalp areas treated with and without Rivastigmine hydrogen tartrate in a formulation. The control means the hair collected from the scalp area treated with the formulation without Rivastigmine hydrogen tartrate.

In an embodiment, the method for quantifying melanin in hair was as described by Fernandes B. et al., 2016 [51]. Hair samples were digested in NaOH and the solutions obtained normalized to 1 mg/mL of hair. Complete oxidation of hair lysates was performed with hydrogen peroxide and melanin content in hair samples calculated by fluoresce spectroscopy using a standard curve generated by the fluorescence of oxidized melanin standards. Melanin change in hair treated with a formulation with Rivastigmine was calculated using the following mathematical formula:

$$\text{Effect} \text{on} \text{melanin} \left(\text{\%}\right) \text{=} \frac{\text{treated} \text{hair} \text{melanin} \text{content}}{\text{control} \text{hair} \text{melanin} \text{content}} \times 100 \text{-} 100$$

The treatment of scalp with the modulation bioactive agent Rivastigmine hydrogen tartrate induced a lightening effect. The brown shade of hair was visibly lighter than the natural shade, after just 5 weeks of treatment (Table 2).

### Example 5

This example discloses the treatment of human scalp with a formulation containing a modulation bioactive agent for darkening the hair.

In an embodiment, the bioactive agent used in this example was Dipyridamole as darkening agent in a formulation containing water, a thickener agent with emulsifying properties - Lecigel™, a preservative agent - Euxyl® PE 9010, denaturing alcohol and Transcutol® CG.

The volunteers for the application test of the formulation with the modulation bioactive agent Dipyridamole had blond hair of different shades.

Each volunteer received the formulation without the agent Dipyridamole and the formulation with the agent Dipyridamole. The formulation that contains the modulation bioactive agent had a concentration of 0.01% (weight by weight) Dipyridamole.

On each volunteer, the hair was shaved in two different posterior scalp locations, of approximately 1 cm² each, and the formulations were applied for 5 weeks, three times a week.

In an embodiment, the formulations with and without Dipyridamole were applied directly on scalp. Ten microliters per 1 cm² of the control formulation without Dipyridamole were applied to one of the two shaved scalp areas while ten microliters per 1 cm² of the formulation with Dipyridamole were applied to the other shaved scalp area.

After the 15^{th} application of formulations with and without the modulation bioactive agent, a final picture was taken, the growth rate of the hairs was measured, and some hairs were plucked for further tests.

The hair melanin quantification process was performed after the clinical cosmetic study conclusion, using hairs collected from the scalp areas treated with and without Dipyridamole in a formulation. The control means hair collected from the scalp area treated with the formulation without Dipyridamole.

The method for quantifying melanin in hair was as described by Fernandes B. et al., 2016 [51]. Hair samples were digested in NaOH and the solutions obtained normalized to 1 mg/mL of hair. Complete oxidation of hair lysates was performed with hydrogen peroxide and melanin content in hair samples calculated by fluoresce spectroscopy using a standard curve generated by the fluorescence of oxidized melanin standards. Melanin change in hair treated with a formulation with Dipyridamole was calculated using the following mathematical formula:

$$\text{Effect} \text{on} \text{melanin} \left(\text{\%}\right) \text{=} \frac{\text{treated} \text{hair} \text{melanin} \text{content}}{\text{control} \text{hair} \text{melanin} \text{content}} \times 100 \text{-} 100$$

The treatment of scalp with the modulation bioactive agent Dipyridamole induced a darkening effect. The brown shade of hair was visibly darker than the natural shade, after just 5 weeks of treatment (Table 2).

TABLE 2

Summary of hair shape and colour modulation results obtained on human volunteers with the exemplified bioactive agents, applied for 5 weeks, 3 times per week
Hair Shape Modulation

| Treatment | | Effectiveness∗ (% of volunteers) | Hair shape change (% of control) |
|---|---|---|---|
| Straightening | Midodrine HCl (0.25%) | 75.0% | +76.0% (máx. +120.0%) |
| Curling | Topiramate (1.75%) | 83.3% | -10.0% (máx. -11.0%) |
| ∗Mean value of the volunteers with effective changes in hair straightening/curling degree. | | | |
| Hair Colour Modulation | | | |
| Treatment | | Effectiveness∗ (% of volunteers) | Hair Melanin change (% of control) |
| Lightening | Rivastigmine (0.2%) | 50.0 | - 19.3% (max. -26.3%) |
| Darkening | Dipyridamole (0.01%) | 67.0 | + 36.0% (max. +57.9%) |

∗Mean value of the volunteers with effective changes in hair melanin content.

The disclosure is of course not in any way restricted to the embodiments described and a person with ordinary skill in the art will foresee many possibilities to modifications thereof without departing from the basic idea of the disclosure as defined in the appended claims.

In the present disclosure, hair includes human hair, human scalp hair, animal hair and animal fur.

In the present disclosure, agents include elements, substances, compounds, molecules, ingredients.

In the present disclosure, the terms composition and formulation are frequently used interchangeably. These terms refer to a mixture of excipients intended to be used in the topical application of active agents, said mixture exhibiting a colour, a smell and a texture that do not generate inacceptable discomfort to the user, such as itching, tightness and redness of the skin (particularly the skin of the scalp).

In the present disclosure, topical or topically refers to directly laying on or spreading on outer skin, especially the scalp, e.g., by use of the hands or an applicator such as a wipe, roller, or spray.

In the present disclosure, the terms cosmetically acceptable, pharmacologically acceptable, pharmaceutically acceptable and dermatologically acceptable means that the term describe ingredients that are suitable for use in contact with tissues (e.g., the skin) of a mammal, preferably of human, without undue toxicity, incompatibility, instability, irritability, allergic response or the like.

In the present disclosure, effective amount means an amount of a physiologically bioactive agent or composition sufficient to induce a change in the hair colour and/or shape but low enough to avoid side effects.

Where singular forms of elements or features are used in the specification of the claims, the plural form is also included, and vice versa, if not specifically excluded. For example, the term “an agent” or “the agent” also includes the plural forms “agents” or “the agents”, and vice versa. In the claims, articles such as “a”, “an”, and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. Moreover, the invention also includes embodiments in which more than one, or all the group members are present in, employed in, or otherwise relevant to a given product or process.

Furthermore, it is to be understood that the invention encompasses all variations, combinations, and permutations in which one or more limitations, elements, clauses, descriptive terms, etc., form one or more of the claims or from relevant portions of the description is introduced into another claim. For example, any claim that is dependent on another claim can be modified to include one or more limitations found in any other claim that is dependent on the same base claim.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

The term “comprising” whenever used in this document is intended to indicate the presence of stated features, integers, steps, components, but not to preclude the presence or addition of one or more other features, integers, steps, components or groups thereof.

The disclosure should not be seen in any way restricted to the embodiments described and a person with ordinary skill in the art will foresee many possibilities to modifications thereof. The above described embodiments are combinable.

The following claims further set out particular embodiments of the disclosure.

### REFERENCES

1. Koch SL, Tridico SR, Bernard BA, et al (2020) The biology of human hair: A multidisciplinary review. Am J Hum Biol, 32:e23316
2. Cruz C, Costa C, Gomes A, et al (2016) Human Hair and the Impact of Cosmetic Procedures: A Review on Cleansing and Shape-Modulating Cosmetics. Cosmetics 3:26
3. Westgate GE, Botchkareva N V, Tobin DJ (2013) The biology of hair diversity. Int J Cosmet Sci, 35:329
4. Neuser F, Schlatter H (2010) Chapter 30: Hair dyes. In: Cosmetic Dermatology: Products and Procedures, Draelos ZD (Ed.), Wiley-Blackwell, pp. 227-235
5. Westgate GE, Ginger RS, Green MR (2017) The biology and genetics of curly hair. Exp Dermatol 26:483
6. Schwan-Jonczyk A, Sendelbach G (2010) Chap. 31: Permanent hair waving. In: Cosmetic Dermatology: Products & Procedures, Draelos ZD(Ed.), Wiley-Blackwell, pp. 236-247
7. Bryant H et al.(2010) Chapter 32: Hair straightening. In: Cosmetic Dermatology: Products & Procedures, Draelos ZD(Ed.), Wiley-Blackwell, pp. 248-255
8. Bhushan B (2010) Introduction: Human Hair, Skin, and Hair Care Products. In: Bhushan B (ed) Biophysics of Human Hair. Springer-Verlag, Berlin, Heidelberg, pp 1-19
9. Wong M, Wis-Surel G, Epps J (1994) Mechanism of hair straightening. J Cosmet Chem 45:347
10. Robbins CR (2012) Chemical and Physical Behavior of Human Hair, 5th ed. Springer - Verlag, New York
11. Seckin D, Yildiz A (2009) Repigmentation and curling of hair after acitretin therapy. Australas J Dermatol 50:214
12. Ward PD, Miller HL, Shipman AR (2014) A case of repigmentation and curling of hair on acitretin therapy. Clin Exp Dermatol 39:91
13. Clarke JT, Price H, Clarke S, George R, Miller JJ (2007) Acquired kinking of the hair caused by acitretin. J Drugs Dermatol 6:937
14. Alting K, van Hunsel F (2018) Curling of hair in two female patients taking alitretinoin. Drug Saf - Case Reports 5:1
15. Rivera N, Boada A, Bielsa MI, et al (2017) Hair repigmentation during immunotherapy treatment with an anti-programmed cell death 1 and anti-programmed cell death ligand 1 agent for lung cancer. JAMA Dermatol 153:1162
16. Donovan JC, Price VH (2010) Chloroquine-induced hair hypopigmentation. N Engl J Med 363:372
17. Di Giacomo TB, Valente NYS, Nico MMS (2009) Chloroquine - induced hair depigmentation. Lupus. 18:264
18. Robinson A, Jones W (1989) Changes in scalp hair after cancer chemotherapy. Eur J Cancer Clin Oncol 25:155
19. Noppakun N, Swasdikul D (1986) Reversible hyperpigmentation of skin and nails with white hair due to vitamin B12 deficiency. Arch Dermatol 122:896
20. Sadighha A, Zahed G. (2008) Hair darkening after treatment with cyclosporin in a patient with psoriasis. J Eur Acad Dermatol Venereol 2:1239
21. Dika E, Patrizi A, Ribero S, et al (2016) Hair and nail adverse events during treatment with targeted therapies for metastatic melanoma. EurJ Dermatol 26:232
22. Fujimi A, Ibata S, Kanisawa Y, et al (2016) Reversible skin and hair depigmentation during chemotherapy with dasatinib for chronic myeloid leukemia. J Dermatol 43:104
23. Rubegni P, Sbano P, Fimiani M (2003) A case of disseminated granuloma annulare treated with defibrotide: complete clinical remission and progressive hair darkening. Br J Dermatol 149:422
24. Cheng Y, Chen H, Chiu H (2014) Erlotinib-induced hair pigmentation. Int J Dermatol 53:55
25. Graham RM, James MP, Ferguson DJP, Guerrier CW (1985) Acquired kinking of the hair associated with etretinate therapy. Clin Exp Dermatol 10:426
26. Nanda A, Alsaleh QA (1994) Hair discoloration caused by etretinate. Dermatology 188:172
27. Nagase K, Inoue T, Narisawa Y (2017) Manifest hair repigmentation associated with etretinate therapy. J Dermatol 44:e34-e35
28. Vesper JL, Fenske NA, Tampa MD (1996) Hair darkening and new growth associated with etretinate therapy. J Am Acad Dermatol 34:860
29. Zheng S, Pan YL, Wang JL, et al (2009) Gefitinib-induced hair alterations. BMJ Case Rep 2009:bcr0920080878
30. Meiler S, Gerber PA, Homey B (2008) Clinical image: blonde by prescription. Arthritis Rheum 58:2286
31. Yun S, Song K, Hwang S, Kim H, Lee N, ParkJ (2014) Hair graying and loss induced by imatinib mesylate. J Dermatol 41:107
32. Mariani S, Abruzzese E, Basciani S, et al (2011) Reversible hair depigmentation in a patient treated with imatinib. Leuk Res 35:64
33. Redondo P, Guzmán M, Marquina M, Pretel M, Lloret LAP, Gorrochategui A (2007) Repigmentation of gray hair after thyroid hormone treatment. Actas Dermosifiliogr 98:603
34. Bellandi S, LAmato L, Cipollini E, Antiga E, Brandini L, Fabbri P (2011) Repigmentation of hair after latanoprost therapy. J Eur Acad Dermatol Venereol 25:2010
35. Dasanu CA (2013) Hair repigmentation associated with the use of lenalidomide : graying may not be an irreversible process ! J Oncol Pharm Pract 19:165
36. Reynolds NJ, Crossley J, Ferguson l, Peachey RDG (1989) Darkening of white hair in Parkinson’s disease. Clin Exp Dermatol 14:317
37. Zarafonetis CJ (1950) Darkening of gray hair during para-amino-benzoic acid therapy. J Invest Dermatol 15:399
38. Sideras K, Menefee ME, Burton JK, Erlichman C, Bible KC, Ivy SP (2010) Profound hair and skin hypopigmentation in an African American woman treated with the multi-targeted tyrosine kinase inhibitor pazopanib. J Clin Oncol 28:312
39. Kobayashi E, Koyama T, Kobayashi K, Setsu N, Kawashima M, Kawai A (2014) Reversible hair depigmentation in a Japanese female treated with pazopanib. J Dermatol 41:1021
40. Calle-Lopez Y, Kotagal P, Knight EP (2019) Perampanel-induced hair curling in a patient with epilepsy associated with Pitt Hopkins syndrome. Epileptic Disord 21:479
41. Dermatol A (2008) Repigmentation of the white hair after systemic corticosteroids for bullous pemphigoid. J Eur Acad Dermatol Venereol 22:1018
42. Hartmann J, Kanz L. (2008) Sunitinib and periodic hair depigmentation due to temporary c-KIT inhibition. Arch Dermatol 144:1525
43. Hampson J, Donnelly A, Lewis-Jones M, Pye J. (1991) Tamoxifen-induced hair colour change. Br J Dermatol 132:483
44. Lovering S, Miao W, Bailie T, Amato D (2016) Hair repigmentation associated with thalidomide use for the treatment of multiple myeloma. BMJ Case Rep 2016.pii: bcr2016215521
45. Karamizadeh Z, Rasekhi AR (2008) Gray hair in children on triptorelin treatment. lnt J Dermatol 47:601
46. GerstnerT, Lipinski C, Longin E, König S (2008) Valproate-induced change in hair color. J Am Acad Dermatol 58:63
47. Yasemin G (2016) Curly hair induced by valproate in bipolar disorder. Clin Psychopharmacol Neurosci 14:114
48. Read G (1991) Verapamil and hair colour change. Lancet 338: 1520
49. Fleming C, MacKie R (1996) Alpha interferon-induced hair discolouration. Br J Dermatol 135:337
50. Luca R De, Trodella M, Tartaro G, Colella G (2013) White tongue and straight hair in a patient with chronic hepatitis C : a case report and review of the literature. Ann Stomatol (Roma) 4:13.eCollection
51. Fernandes B, Matamá T, Guimarães D, Gomes A, Cavaco-Paulo A (2016) Fluorescent quantification of melanin. Pigment Cell Melanoma Res 29:70

1. A composition for hair follicle modulation comprising:
a bioactive agent selected from a group consisting of dipyridamole, dipyridamole salt, rivastigmine, rivastigmine salt, paroxetine, paroxetine salt, ethacrynic acid, ethacrynic salt, midodrine, midodrine salt, topiramate, topiramate salt, entacapone, entacapone salt, or mixtures thereof;
a thickener agent; a preservative, and a denaturing alcohol;
wherein the hair follicle modulation is a hair colour modulation and/or a hair volume modulation;
with the proviso that paroxetine, and/or paroxetine salt are not used for hair volume modulation.

2. The composition according to claim 1, wherein the bioactive agent is selected from a group consisting of: dipyridamole, dipyridamole salt, rivastigmine, rivastigmine salt, ethacrynic acid, ethacrynic salt, midodrine, midodrine salt, topiramate, topiramate salt, entacapone, entacapone salt, or mixtures thereof.

3. The composition according to claim 1, comprising 0.001-50% by weight of the bioactive agent .

4. The composition according to claim 1, wherein the thickener agent is selected from a group consisting of: sodium acrylates copolymer and lecithin; aluminum stearates/isostearates/myristates/laurates/palmitates, glycol distearate, hydrogenated castor oil, hydrogenated castor oil hydroxystearate, hydrogenated castor oil isostearate, hydrogenated castor oil stearate, hydrogenated castor PEG-8 esters, PEG-150 distearate, polyethylene glycol, polyacrylic acid/carbomer, carcomer 934, tea-carbomer, carboxymethyl chitin, carboxymethyl chitosan, carboxymethyl dextran, carboxymethyl hydroxypropyl guar, sodium carbomer, sodium dextran sulfate, sodium polystyrene sulfonate, sodium surfactin, stearalkonium bentonite, stearalkonium hectorite, steareth-30/-40/-50, xanthan gum, vegetable gums, Ahnfeltia confina extract, Euglena gracilis polysaccharide or mixtures thereof.

5. The composition according to claim 1, wherein the preservative agent is selected from a group consisting of: phenoxyethanol and ethylhexylglycerin; butyl paraben, diazolidinyl urea, DMDM hydantoin, ethyl paraben, imidazolidinyl urea, iodopropynyl butylcarbamate, isobutyl paraben, methyl paraben, methylchloroisothiazolinone, methylisothiazolinone, phenoxyethanol, ethylhexylglycerin, propyl paraben, sodium benzoate, Germaben II, Germall Plus, Kathon, potassium sorbate/ sorbic acid, quaternium-15, polyoxymethylene urea, sodium hydroxymethylglycinate, bromopol, glyoxal, isopropylparaben, benzylate, benzoic acid, and benzyl ester, triclosan and triclocarban, benzyl alcohol, benzalkonium chloride, citric acid, dehydroacetic acid, essential oils, grapefruit seed extract, lactic acid, levulinic acid, sodium dehydroacetate, sodium metabisulfite, sodium salicylate, vitamin E, zinc pyrithione, or mixtures thereof.

6. The composition according to claim 1, wherein the denaturing alcohol is selected from a group consisting of: diethylene glycol monoethyl ether; 1,2,6 hexanetriol, dipropylene glycol, glycerin, hexylene glycol, panthenol, phytantriol, propylene glycol, sodium pyroglutamic acid (PCA), sorbitol, diethylene glycol monoethyl ether, triethylene glycol, polyglyceryl sorbitol, glucose, fructose, polydextrose, potassium PCA, hydrogenated honey, hyaluronic acid, inositol, hexanediol beeswax, hexanetriol beeswax, hydrolyzed elastin, hydrolyzed collagen, hydrolyzed silk, hydrolyzed keratin, erythritol, capryl glycol, isoceteth-(3-10, 20, 30), isolaureth-(3-10, 20, 30), laneth-(5-50), laureth-(1-30), steareth-(4-20), trideceth-(5-50), lithium chloride, glyceryl triacetate, butylene glycol, lactic acid, aloe vera, xylitol, maltitol, castor oil, urea, tripropylene glycol, collagen, pentylene glycol, or mixtures thereof.

7. The composition according to claim 1, wherein the hair colour modulation comprises hair darkening or hair lightening.

8. The composition according to claim 1, wherein the hair volume modulation comprises hair straightening or hair curling.

9. The composition according to claim 1, wherein the colour modulation is regulated by dipyridamole, dipyridamole salt, rivastigmine salt, paroxetine, paroxetine salt or mixtures thereof.

10. The composition according to claim 1, wherein hair darkening is regulated by dipyridamole or dipyridamole salt.

11. The composition according to claim 1, wherein hair lightening is regulated by rivastigmine, rivastigmine salt, paroxetine, paroxetine salt or mixtures thereof.

12. The composition according to claim 1, wherein the hair volume modulation is regulated by ethacrynic acid, ethacrynic salt, midodrine, midodrine salt, topiramate, topiramate salt, entacapone, entacapone salt, or mixtures thereof.

13. The composition according to claim 1, wherein hair straightening is regulated by ethacrynic acid, ethacrynic salt, midodrine, midodrine salt, or mixtures thereof.

14. The composition according to claim 1, wherein hair curling is regulated by topiramate, topiramate salt, entacapone, entacapone salt, or mixtures thereof.

15. The composition according to claim 1, wherein hair modulation comprises hair darkening and hair straightening.

16. The composition according to claim 1, wherein hair modulation comprises hair darkening and hair curling.

17. The composition according to claim 1, wherein hair modulation comprises hair lightening and hair straightening.

18. The composition according to claim 1, wherein hair modulation comprises hair lightening and hair curling.

19. The composition according to claim 1, wherein the bioactive agent is encapsulated or bonded to a controlled release system, in particular a liposome.

20. The composition according to claim 1, wherein the composition is a topical composition.

21. The composition according to claim 20, wherein the composition is administered topically to the skin of a mammal, preferably the skin of the human scalp.

22. The composition according to claim 21, wherein the topical composition is a solution, hydroalcoholic solution, dispersion, suspension, tonic, emulsion, lotion, elixir, serum, toner, cleanser, cream, mask, mousse, ointment, gel, wax, oil, foam, soap, shampoo, conditioner, spray, aerosol, powder, paste.

23. The composition according to claim 1, further comprising at least one cosmetically and/or pharmaceutically and/or dermatologically acceptable excipient.

24. The composition according to claim 1, further comprising at least one of the excipients and/or compounds selected from the following list: surfactants, anionic surfactant, amphoteric surfactant, cationic surfactant, nonionic surfactant emulsifiers, preservatives, thickeners, natural polymers derivative, organic polymers, proteins, humectants, cationic polymers, silicones, oils (including organic and natural oils), fragrances, vitamins, emollient esters, alkanolamides, amines, buffers, pH adjustors, salts, antimicrobial agents, antibacterial agents, aliphatic alcohols, UV filters/sunscreens, amine oxides, chelates, fatty acids, PEG-modified materials, polymers, anti-static agents, alcohols, disinfectants agents, or any mixture thereof.

25. The composition according to claim 1, for use in the prevention, therapy or treatment of abnormal defective production of melanin.

26. The composition according to claim 1, for use as self-tanner agent.

27. A pharmaceutical composition for use in the prevention, therapy or treatment of abnormal defective production of melanin, comprising a bioactive agent selected from a group consisting of: dipyridamole, dipyridamole salt, rivastigmine, rivastigmine salt, paroxetine, paroxetine salt, or mixtures thereof.

28. The pharmaceutical composition according to claim 27, for use in the prevention, therapy or treatment of melasma; solar or senile lentigo, freckles due to abnormal excessive production of melanin, post-medicinal hyperpigmentation, post-inflammatory hyperpigmentation, light induced hyperpigmentation and chemical induced hyperpigmentation.

29. The pharmaceutical composition according to claim 27, further comprising a pharmaceutical acceptable thickener agent; a pharmaceutical acceptable preservative; and a pharmaceutical acceptable denaturing alcohol.

30. The pharmaceutical composition according to claim 27, wherein the bioactive agent is encapsulated or bonded to a controlled release system, in particular a liposome.

31. Use of a bioactive agent as a hair modulator wherein the bioactive agent is selected from a group consisting of: dipyridamole, dipyridamole salt, rivastigmine, rivastigmine salt, paroxetine, paroxetine salt, ethacrynic acid, ethacrynic salt, midodrine, midodrine salt, topiramate, topiramate salt, entacapone, entacapone salt, or mixtures thereof, wherein the hair modulator is a hair colour modulator and/or a hair volume modulator; wherein paroxetine, and/or paroxetine salt are not use as hair volume modulator.

32. Use of a bioactive agent as a hair modulator wherein the bioactive agent is selected from a group consisting of: dipyridamole, dipyridamole salt, rivastigmine, rivastigmine salt, paroxetine, paroxetine salt, ethacrynic acid, ethacrynic salt, midodrine, midodrine salt, topiramate, topiramate salt, entacapone, entacapone salt, or mixtures thereof.

33. Use of a bioactive agent according to claim 31, wherein the hair modulator is a colour modulator and/or a volume modulator.

34. Use of bioactive agent according to claim 31, wherein colour modulator is a darkener or lightener of a hair fiber.

35. Use of a bioactive agent according to claim 31, wherein hair volume modulator is a hair straightener or hair curler.

36. Use of a bioactive agent according to claim 31, wherein the bioactive agent is encapsulated or bonded to a controlled release system, in particular a liposome.

37. A kit, cosmetic product, or reagent comprising the composition described by claim 1.
